(19) United States
(12) Patent Application Publication
(10) Pub. No.: US 2026/0335270 A1
(43) Pub. Date: Oct. 29, 2026

(54) METHODS AND COMPOUNDS FOR TREATING ULCERATIVE COLITIS, SYSTEMIC LUPUS ERYTHEMATOSUS, SEPSIS, AND POST-SEPSIS SYNDROME

(71) Applicant: Redox Bioscience LLC, Palm Beach Gardens, FL (US)

(72) Inventor: Jay Pravda, Palm Beach Gardens, FL (US)

(21) Appl. No.: 19/099,011

(22) PCT Filed: Jul. 10, 2023
(86) PCT No.: PCT/US2023/069869

§ 371 Date: Jan. 27, 2025

Related U.S. Application Data
(60) Provisional application No. 63/369,539, filed on Jul. 27, 2022.

(51) Int. Cl. A61K 31/20; A61K 9/00; A61K 31/19; A61K 47/02; A61K 47/10; A61K 47/14; A61K 47/22; A61K 47/24; A61K 47/42; A61K 47/44; A61K 47/46; A61P 1/04

(52) U.S. Cl. CPC A61K 31/20; A61K 9/0053; A61K 31/19; A61K 47/02; A61K 47/10; A61K 47/14; A61K 47/22; A61K 47/24; A61K 47/42; A61K 47/44; A61K 47/46; A61P 1/04

(57) ABSTRACT

The present inventive concept provides methods for treating or preventing ulcerative colitis, systemic lupus erythematosus (SLE), sepsis, or post-sepsis syndrome including administering a therapeutically effective amount of dihydro-lipoic acid, or a derivative, pharmaceutically acceptable salt, metabolite, solvate, prodrug, enantiomer, or polymorph of the dihydro-lipoic acid. The inventive concept further provides compositions and formulations for uses described herein.

### RELATED APPLICATION

The present application claims the benefit of U.S. Provisional Patent Application Ser. No. 63/369,539, filed Jul. 27, 2022, the disclosures of which is incorporated herein by reference.

### COPYRIGHT

This disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD

The present disclosure generally relates to methods of treating and preventing ulcerative colitis, systemic lupus erythematosus (SLE), sepsis, and/or post-sepsis syndrome in a subject in need thereof; and, more particularly, to methods that include administering a composition including a therapeutically effective amount of dihydro-lipoic acid, which includes, derivatives, pharmaceutically acceptable salts, metabolites, solvates, prodrugs, enantiomers, or polymorphs of the dihydro-lipoic acid, to a subject in need thereof. Compositions for treating and preventing ulcerative colitis, systemic lupus erythematosus, sepsis, and/or post-sepsis syndrome are also provided.

### BACKGROUND

Ulcerative colitis is an inflammatory bowel disease characterized by recurrent bouts of rectal bleeding and bloody diarrhea. Ulcerative colitis has been characterized as a two-phase disease. The first phase is called induction and begins with the extracellular diffusion of hydrogen peroxide to the extracellular environment. The induction phase occurs prior to any colonic bleeding, which may last for months to years. The second phase of ulcerative colitis is called the propagation phase and begins with neutrophilic invasion into the colonic tissues. The propagation phase begins with rectal bleeding. Further information about ulcerative colitis is disclosed in U.S. Pat. No. 8,476,233 (Jul. 2, 2013); U.S. Pat. No. 8,916,546 (Dec. 23, 2014); U.S. Pat. No. 9,511,049 (Dec. 6, 2016); and U.S. Pat. No. 10,064,877 (Sep. 4, 2018); and Pravda J. Evidence-based pathogenesis and treatment of ulcerative colitis: A causal role for colonic epithelial hydrogen peroxide. World J Gastroenterol 2022 August; 28(31): 4263-4298, the contents of which are hereby incorporated by reference in their entireties, including the portions discussing ulcerative colitis and all other portions.

Existing treatments for ulcerative colitis include the administration of an enema composition or an immunosuppressive agent to the subject in need thereof. Methods of treating ulcerative colitis that include the administration of an enema composition can cause undesired side effects in subjects and/or suffer from poor compliance by the subjects due to the inconvenience of the administration route. Methods of treatment that include immunosuppressive therapeutics fail to achieve long lasting histologic remission of ulcerative colitis, which is characterized by the absence of active inflammation (i.e., no neutrophils) upon examination using microscopic biopsy. For at least these reasons, there remains a need for additional therapeutic compositions and methods of treating ulcerative colitis that can minimize undesired side effects, increase patient compliance with the treatment protocol, and achieve histologic remission of ulcerative colitis.

Similarly, there are other chronic conditions such as systemic lupus erythematosus (SLE), sepsis, post-sepsis syndrome, diabetes, Alzheimer's disease, Parkinson's disease, amyotrphic lateral sclerosis (ALS), autism, psoriasis, cataracts, and atrial fibrillation for which there remain a need for additional therapeutic compositions and methods of treating these disorders that can minimize undesired side effects, increase patient compliance with the treatment protocol, and achieve long-term remission.

### SUMMARY

The present disclosure relates to methods and compositions for treating or preventing ulcerative colitis, systemic lupus erythematosus (SLE), sepsis, or post-sepsis syndrome.

Embodiments of the present inventive concept include methods of treating or preventing ulcerative colitis, systemic lupus erythematosus (SLE), sepsis, or post-sepsis syndrome including administering a therapeutically effective amount of dihydro-lipoic acid, which includes a derivative, pharmaceutically acceptable salt, metabolite, solvate, prodrug, or polymorph of the dihydro-lipoic acid, to a subject in need of such treatment.

Embodiments of the present inventive concept also include methods of reducing the risk or incidence of colectomy or ulcerative colitis-associated colorectal cancer including administering a therapeutically effective amount of dihydro-lipoic acid, which includes derivatives, pharmaceutically acceptable salts, metabolites, solvates, prodrugs, enantiomers, or polymorphs of the dihydro-lipoic acid, to a subject in need of such treatment.

Embodiments of the present inventive concept include methods of treating or preventing a neurodegenerative disorder including administering a therapeutically effective amount of dihydro-lipoic acid, or a derivative, pharmaceutically acceptable salt, metabolite, solvate, prodrug, enantiomer, or polymorph of the dihydro-lipoic acid, to a subject in need of such treatment.

Embodiments of the present inventive concept also include methods of treating or preventing a condition associated with an excessive buildup of hydrogen peroxide including administering a therapeutically effective amount of dihydro-lipoic acid, or a derivative, pharmaceutically acceptable salt, metabolite, solvate, prodrug, enantiomer, or polymorph of the dihydro-lipoic acid, to a subject in need of such treatment.

Embodiments of the present inventive concept further include methods of restoring colonic redox homeostasis by reducing colonic epithelial hydrogen peroxide, the methods including administering a therapeutically effective amount of dihydro-lipoic acid, which includes derivatives, pharmaceutically acceptable salts, metabolites, solvates, prodrugs, enantiomers, or polymorphs of the dihydro-lipoic acid.

Embodiments of the present inventive concept include methods of reversing hydrogen peroxide induced mitochondrial DNA oxidative damage and/or mitochondrial heteroplasmy including administering a therapeutically effective amount of dihydro-lipoic acid, which includes derivatives, pharmaceutically acceptable salts, metabolites, solvates, prodrugs, enantiomers, or polymorphs of the dihydro-lipoic acid.

Embodiments of the present inventive concept further include novel compositions including dihydro-lipoic acid, which includes a derivative, pharmaceutically acceptable salt, metabolite, solvate, prodrug, enantiomer, or polymorph of the dihydro-lipoic acid; a medium-chain triglyceride and/or long-chain triglyceride; a protein derived from collagen; a sugar alcohol; water; at least one glycerophospholipid; at least one fat soluble vitamin; a silicon-based compound; optionally a preservative; and optionally a flavoring.

Embodiments of the present inventive concept further include the compounds and compositions described herein for the preparation and use as a medicament as well as for use in the treatment of the conditions described herein.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings assist with illustrating the various embodiments described herein and, together with the description, explain the principles and operations of the claimed subject matter.

### BRIEF DESCRIPTION OF THE DRAWINGS

A complete understanding of the present embodiments and the advantages and features thereof will be more readily understood by reference to the following detailed description, appended claims, and accompanying drawings, wherein:

FIG. 1 provides the structures of chemical Formulae I-VI, in accordance with embodiments described herein; and

FIG. 2 provides the structures of chemical compounds (a)-(h), in accordance with embodiments described herein.

The drawings are not necessarily to scale, and certain features and certain views of the drawings may be shown exaggerated in scale or in schematic in the interest of clarity and conciseness.

### DETAILED DESCRIPTION

Reference will now be made in detail to the exemplary embodiment(s), examples of which is/are illustrated in the examples. Before describing the exemplary embodiments, it is noted that the embodiments reside primarily in combinations of components and procedures related to the treatment of ulcerative colitis. Accordingly, the composition and method components have been represented where appropriate, showing only those specific details that are pertinent to understanding the embodiments of the present disclosure so as not to obscure the disclosure with details that will be readily apparent to those of ordinary skill in the art having the benefit of the description herein.

The specific details of the various embodiments described herein are used for demonstration purposes only, and no unnecessary limitation or inferences are to be understood therefrom. For example, references to “various embodiments,” “some embodiments,” “other embodiments,” “an example embodiment,” etc., indicate that the embodiment described may include a particular feature, structure, or characteristic, but every embodiment may not necessarily include that feature, structure, or characteristic. Such phrases are not necessarily referring to the same embodiment. When a particular feature, structure, or characteristic is described in connection with an embodiment, it is within the knowledge of one skilled in the art to affect such feature, structure, or characteristic in connection with other embodiments whether explicitly described or not.

A recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. As will be understood by one skilled in the art, ranges disclosed herein encompass any and all possible subranges and combinations of subranges thereof. Any listed range can be easily recognized as sufficiently describing the same range being broken down into at least equal halves, thirds, quarters, fifths, tenths, etc. As a non-limiting example, each range discussed herein can be readily broken down into a lower third, middle third and upper third, etc. As will also be understood by one skilled in the art, language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a composition having 1 to 3 components refers to compositions having 1, 2, or 3 components. Similarly, a composition having 1 to 5 components refers to compositions having 1, 2, 3, 4, or 5 components, and so forth.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, the use of examples, or exemplary language (e.g., “such as”), is intended to illuminate the embodiments and does not pose a limitation on the scope of the claims unless otherwise stated. No language in the specification should be construed as indicating any non-claimed element as essential.

As used herein, the terms “about” and “substantially” will be understood by persons of ordinary skill in the art and will vary to some extent depending upon the context in which it is used. If there are uses of the term which are not clear to persons of ordinary skill in the art, given the context in which it is used, “about” and “substantially” will mean up to plus or minus 10% of the particular term.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of the inventive concept is not intended to be interpreted to be equivalent to “comprising.”

Nonetheless, in some embodiments, “comprise” may also encompass, and, in some embodiments, may refer to the expressions “consist essentially of” and/or “consist of.” Thus, the expression “comprise” can also refer to and encompass embodiments, wherein that which is claimed “comprises” specifically listed elements does not include further elements, as well as embodiments wherein that which is claimed “comprises” specifically listed elements may and/or does encompass further elements, or encompass further elements that do not materially affect the basic and novel characteristic(s) of that which is claimed. For example, that which is claimed, such as a method, kit, system, etc. “comprising” specifically listed elements also encompasses, for example, a method, kit, system, etc. “consisting of,” i.e., wherein that which is claimed does not include further elements, and, for example, a method, kit, system, etc. “consisting essentially of,” i.e., wherein that which is claimed may include further elements that do not materially affect the basic and novel characteristic(s) of that which is claimed.

In various embodiments, methods of treating or preventing ulcerative colitis, systemic lupus erythematosus (SLE), sepsis, or post-sepsis syndrome are provided. In some embodiments, methods of treating or preventing ulcerative colitis, systemic lupus erythematosus, sepsis, or post-sepsis syndrome comprise, consist essentially of, or consist of administering a therapeutically effective amount of dihydro-lipoic acid. Such methods include administering derivatives, pharmaceutically acceptable salts, metabolites, solvates, prodrugs, enantiomers, or polymorphs of the dihydro-lipoic acid. Further methods include administering a composition comprising, consisting essentially of, or consisting of a therapeutically effective amount of dihydro-lipoic acid to a subject in need of such treatment. The compositions include derivatives, pharmaceutically acceptable salts, metabolites, solvates, prodrugs, enantiomers, or polymorphs of the dihydro-lipoic acid.

In some embodiments, the composition including the therapeutically effective amount of dihydro-lipoic acid, which includes derivatives, pharmaceutically acceptable salts, metabolites, solvates, prodrugs, enantiomers, or polymorphs of the dihydro-lipoic acid, is formulated for oral administration. In particular embodiments, the formulation is in capsular form. According to some embodiments, the methods do not include the co-administration of an enema composition to the subject. In still other embodiments, the methods do not include the co-administration of an immunosuppressive agent to the subject. In some embodiments, the dihydro-lipoic acid is co-administered with sodium butyrate. In particular embodiments, the sodium butyrate is administered through colonic release.

In some embodiments, the methods include administration of dihydro-lipoic acid, which includes derivatives, pharmaceutically acceptable salts, metabolites, solvates, prodrugs, or polymorphs of the dihydro-lipoic acid or a composition including a therapeutically effective amount of the same, to the subject during the induction phase of ulcerative colitis.

According to some embodiments of the present inventive concept, the methods include administration of dihydro-lipoic acid to a subject who suffers from severe ulcerative colitis. In some embodiments, the subject suffers from acute ulcerative colitis. In still other embodiments, the subject suffers from chronic ulcerative colitis. In particular embodiments, the subject is asymptomatic for ulcerative colitis. Some asymptomatic subjects may present histologic inflammatory features of ulcerative colitis.

### Ulcerative Colitis.

Ulcerative colitis is a form of inflammatory bowel disease that follows a chronic relapsing and remitting course characterized by abdominal pain, bloody diarrhea, urgency, and tenesmus, all of which are related to inflammation of the large intestine. An immune abnormality is currently the generally accepted mechanism thought to cause this condition despite extensive research conducted since the mid-twentieth century having failed to establish a primary antecedent immune abnormality in individuals with ulcerative colitis or their family members. More recently, however, significantly elevated levels of hydrogen peroxide (H₂O₂) have been documented in the colonic epithelium prior to the appearance of inflammation in individuals with ulcerative colitis, which suggests a causal role in the development of the disease.

Hydrogen peroxide is produced by every cell in the human body and has an important physiological role in cellular processes such as membrane signal transduction, gene expression, cell differentiation, insulin metabolism, cell shape determination, and growth factor induced signaling cascades. When produced in excess, however, cellular hydrogen peroxide has been implicated in the development of ulcerative colitis after significantly elevated colonic mucosal hydrogen peroxide (a known colitic agent) were reported prior to the appearance of colonic inflammation in patients with ulcerative colitis.

Excess hydrogen peroxide produced by colonocytes (colonic epithelial cells) can diffuse through the cell membrane to the extracellular space. Hydrogen peroxide's unique properties of cell membrane permeability, long life, potent oxidizing potential, and neutrophilic chemotactic ability combine to promote oxidative disintegration of colonic epithelial tight junctional proteins while attracting white blood cells into the colonic epithelium, both of which lead to colonic inflammation and eventual ulcerative colitis.

As used herein and as understood by those of ordinary skill in the art, ulcerative colitis can be categorized by degree of severity. For example, mild ulcerative colitis is typically considered to be fewer than 5 bowel movements per day, and does not negatively affect daily life, as patients are able to adapt to the bowel frequency.

Moderate to severe UC is characterized by more frequent bowel movements, such as up to 10 per day accompanied by bleeding.

Acute severe ulcerative colitis (ASUC) refers to a condition defined by the modified Truelove and Wits criteria and combines presence of bloody stools ≥6 times a day with symptoms of systemic toxicity.

Chronic ulcerative colitis refers to ulcerative colitis that lasts 1 year or more and requires ongoing medical attention and/or limits activities of daily living.

Refractory unresponsive ulcerative colitis or refractory ulcerative colitis refers to moderate to severe disease wherein subjects are dependent on or refractory to corticosteroids, have severe endoscopic disease activity (presence of ulcers), or are at high risk of colectomy.

Based upon the neutralizing effect on hydrogen peroxide and the reduction-oxidation (redox) effect on mitochondrial DNA (mtDNA) attributed to the compounds and compositions of the present disclosure, embodiments of the present inventive concept also include methods of reducing the risk or incidence of colectomy or ulcerative colitis-associated colorectal cancer; treating and preventing ulcerative colitis-associated ileitis; restoring colonic redox homeostasis by reducing colonic epithelial hydrogen peroxide; achieving long-term histologic remission for ulcerative colitis or complete epithelial mucosal healing of epithelial tissue, particularly of the gastrointestinal tract; and reversing hydrogen peroxide induced mitochondrial DNA oxidative damage and/or mitochondrial heteroplasmy. Such methods include administering a therapeutically effective amount of dihydro-lipoic acid, which includes a derivative, pharmaceutically acceptable salt, metabolite, solvate, prodrug, or polymorph of the dihydro-lipoic acid, to a subject in need of such treatment.

Moreover, when produced in excess, cellular hydrogen peroxide has been implicated in the development or proliferation of other diseases, including systemic lupus erythematosus, sepsis, and post-sepsis syndrome.

### Systemic Lupus Erythematosus.

Systemic lupus erythematosus (“lupus”) is believed to be caused by the release of autoantigenic molecules (e.g., DNA, histones) into an extracellular environment where they are recognized by the immune system, which undergoes systemic activation causing the release of inflammatory cytokines and the generation of auto-antibodies that can lead to muscle, joint pain, fever, rashes, and/or kidney failure. White blood cells (lymphocytes) are the cells that most often release DNA. When stimulated by an infection, lymphocytes undergo clonal expansion and become hypermetabolic, which generates a large amount of hydrogen peroxide via the electron transport chain. The hydrogen peroxide produced in this process consumes the reducing agents present in the cells (e.g., glutathione). If cells cannot eliminate the hydrogen peroxide, then excess hydrogen peroxide will accumulate in the blood stream and trigger apoptosis, which will release DNA from the cells into systemic circulation. Another common source of DNA release originates from skin cells when a subject is exposed to ultraviolet (UV) radiation from the sun. Exposure to UV rays generates oxygen radicals and hydrogen peroxide, which triggers apoptosis causing a subsequent release of DNA, which leads to de-novo lupus or a lupus flare in previously diagnosed patients.

### Sepsis.

Sepsis can occur when hydrogen peroxide builds up in the blood stream subsequent to a generalized hypermetabolic reaction. Such reactions are usually due to a generalized bacterial infection in the blood (septicemia). Hydrogen peroxide is initially released from muscle and lung cells, but it can also be released from other cells in the body once existing reducing agents are used up. Hydrogen peroxide is cell membrane permeable and causes organ failure by inhibiting enzymes and other molecules involved in mitochondrial cellular bioenergetics. Hydrogen peroxide can lead to a decrease in ATP levels and organ failure. Hydrogen peroxide also activates the coagulation cascade causing disseminated intravascular coagulation. Hydrogen peroxide damages endothelial cells leading to refractory hypotension and shock. Any non-infectious insult that causes a generalized hypermetabolic reaction can lead to increased hydrogen peroxide production, consumption of cellular glutathione, and sepsis (i.e., pancreatitis, pulmonary embolism). Sepsis can also occur after a major surgery or serious accident with severe generalized body trauma leading to a generalized hypermetabolic state with supraphysiologic production of hydrogen peroxide followed by consumption of cellular reductive (antioxidant) capacity and the appearance of free hydrogen peroxide in the blood resulting in sepsis.

### Post-Sepsis Syndrome.

Certain sepsis survivors can develop a post-sepsis syndrome, which is also referred to as post-intensive care syndrome because it can occur in any acutely ill patient admitted to the intensive care unit (ICU). During an acute period in the ICU, a hypermetabolic state can generate high amounts of hydrogen peroxide, which can permeate through cells of the body and cause the oxidation of mitochondrial DNA (mtDNA). Oxidized mtDNA can cause problems that lead to a substantial increase in mortality and morbidity in the five years after discharge from the ICU. Approximately half of those patients end up with long term neuromuscular problems (weakness), cognitive problems, and/or a variety of other problems that increase morbidity and mortality. One problem is that damage to mtDNA decreases the synthesis of ATP in organs. This can cause dysfunction in any organ where mtDNA damage has occurred. Another problem is an increased generation of hydrogen peroxide by the electron transport chain in cells with damaged mtDNA. The increased concentration of cellular hydrogen peroxide can cause bioenergetic failure. The combination of these effects will generally lead to significantly increased morbidity and mortality for survivors of acute illness after they are discharged from the ICU (i.e., sepsis, multiple body trauma, etc.).

As used herein, the terms “subject,” “individual,” or “patient” include, but are not limited to avian, vertebrate and mammalian species. Illustrative avians according to the present disclosure include chickens, ducks, turkeys, geese, quail, pheasant, ratites (e.g., ostrich) and domesticated birds (e.g., parrots and canaries), and birds in ovo.

Mammals of the present disclosure include, but are not limited to, canines, felines, bovines, caprines, equines, ovines, porcines, rodents (e.g. rats and mice), lagomorphs, primates (including non-human primates), humans, and the like. According to some embodiments of the present disclosure, the mammal is a non-human mammal (living or deceased). In some embodiments, the mammal is a human subject (living or deceased). Human subjects of both genders are suitable subjects according to the present invention. Further, the subjects relevant to this invention may be male or female and may be any age such as less than 12 months to over 100 including newborns, infants, juveniles, adolescents, teenagers, adults and geriatrics. Subjects relevant to this disclosure further include any species and may be of any race or ethnicity, including, but not limited to, Caucasian, African-American, African, Asian, Hispanic, Indian, etc., and combined backgrounds.

Particularly relevant subjects to this inventive concept are subjects who suffer from ulcerative colitis or are susceptible to ulcerative colitis. Other subjects include those who have failed medical therapy for ulcerative colitis. Still others include those having risk factors for ulcerative colitis including, but not limited to, those with a family history of ulcerative colitis; those who have experienced uncontrolled inflammation in the gastrointestinal tract as a reaction to environmental factors such as bacteria or chemicals, for example, mercury or perfluorooctanoic acid (PFOA) exposure; alcohol use; those having high-fat and/or low-fiber diets; antibiotic use; exposure to oxidative stressors; psychological stress; and cigarette smoking.

Other subjects of interest include those who suffer from systemic lupus erythematosus (SLE), sepsis, or post-sepsis syndrome ulcerative colitis or are susceptible to systemic lupus erythematosus, sepsis, or post-sepsis syndrome. Other subjects include those who have failed medical therapy for these disorders. Still others include those having risk factors for systemic lupus erythematosus, sepsis, or post-sepsis syndrome.

The methods disclosed herein can also be carried out on animal subjects, particularly mammalian subjects such as mice, rats, dogs, cats, livestock and horses for veterinary treatment or prevention purposes, and for screening and development purposes.

The term “administering” a composition to a subject means delivering the composition to the subject. “Administering” can include prophylactic administration of the composition (i.e., before one or more symptoms of an inflammatory condition are detectable) and/or therapeutic administration of the composition (i.e., after the inflammatory condition and/or one or more symptoms of the inflammatory condition are detectable). The methods of some embodiments may include administering one or more compounds, compositions, or agents. If more than one compound, composition, or agent is to be administered it may be administered together at substantially the same time, and/or be administered before, concomitantly with, and/or after administration of another composition or therapeutic procedure.

As used herein, the terms “effective amount” or “therapeutically effective amount,” refer to a quantity sufficient to achieve a desired therapeutic and/or prophylactic effect, e.g., an amount which results in the full or partial amelioration of disorders or symptoms associated with ulcerative colitis or other described herein in a subject. In the context of therapeutic or prophylactic applications, the amount of a composition administered to the subject will depend on the type and severity of the disorder or symptom and on the characteristics of the individual, such as general health, age, sex, body weight, and tolerance to drugs. It will also depend on the degree, severity, and type of disorder or symptom. The compositions can also be administered in combination with one or more additional compounds or compositions. In some embodiments, multiple doses are administered. In some embodiments, multiple therapeutic compositions or compounds are administered. In the methods described herein, the compositions may be administered to a subject having one or more signs or symptoms of a disorder described herein.

Moreover, by the terms “treat,” “treating” or “treatment of,” it is intended that the severity of the disorder or the symptoms of the disorder are reduced, or the disorder is partially or entirely eliminated, as compared to that which would occur in the absence of treatment. Treatment does not require the achievement of a complete cure of the disorder.

By the terms “preventing” or “prevention,” it is intended that the inventive methods eliminate or reduce the incidence or onset of the disorder, as compared to that which would occur in the absence of the measure taken. Alternatively stated, the present methods slow, delay, control, or decrease the likelihood or probability of the disorder in the subject, as compared to that which would occur in the absence of the measure taken.

### Compounds.

Dihydro-lipoic acid is the reduced form of lipoic acid. The natural function of lipoic acid (e.g., alpha-lipoic acid) is to bind and transfer acyl groups to successive enzymatic active sites among the subunits of each enzyme complex. In the acyl transfer process, lipoic acid is reduced to dihydro-lipoic acid and subsequently re-oxidized back to lipoic acid by a cognate enzyme, which readies it for the next acyl transfer. The structure of dihydro-lipoic acid, shown below, includes an eight carbon chain substituted at the C1 position with a carboxylic acid and at each of the C6 and C8 positions with a thiol. The chemical name of dihydro-lipoic acid is 6,8-Bis(sulfanyl)octanoic acid.

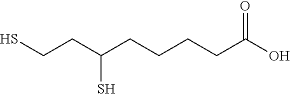 [SMILES: O=C(O)CCCCC(S)CCS]

### Dihydro-Lipoic Acid:

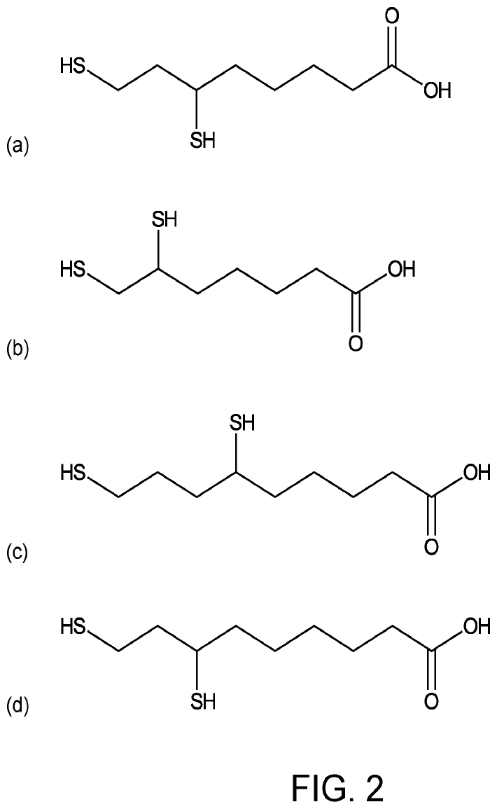

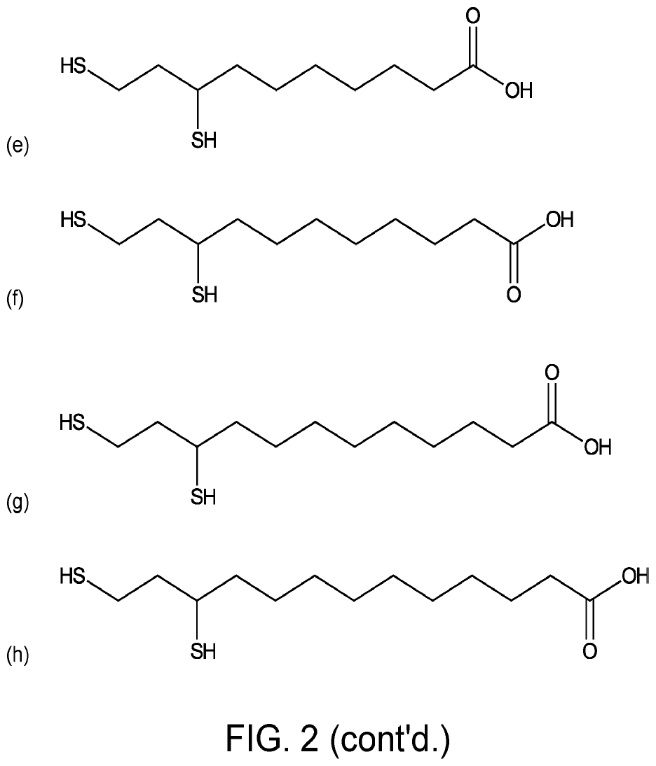

Dihydro-lipoic acid is referenced as compound (a) as shown in FIG. 2.

In some embodiments, the method comprises administering a composition comprising dihydro-lipoic acid to a subject in need thereof. In some embodiments, the composition includes racemic dihydro-lipoic acid, the R-enantiomer of dihydro-lipoic acid, the S-enantiomer of dihydro-lipoic acid, pharmaceutically acceptable salts thereof, or any suitable combination thereof. In some embodiments, the composition comprises R-dihydro-lipoic acid or a pharmaceutically acceptable salt thereof. In some embodiments, the method does not include the S-enantiomer of dihydro-lipoic acid. In some embodiments, the method does not include the co-administration of an enema composition to the subject. In some embodiments, the method does not include the co-administration of an immunosuppressive agent to the subject. In some embodiments, the method comprises administration of a composition comprising a therapeutically effective amount of dihydro-lipoic acid or R-dihydro-lipoic acid to the subject during the induction phase of ulcerative colitis. In some embodiments, the method comprises administration of a composition comprising a therapeutically effective amount of dihydro-lipoic acid or R-dihydro-lipoic acid to the subject diagnosed with systemic lupus erythematosus (SLE), sepsis, and/or post-sepsis syndrome. In some embodiments, the method comprises administration of a composition comprising a therapeutically effective amount of dihydro-lipoic acid or R-dihydro-lipoic acid to the subject diagnosed with a neurodegenerative disorder, including, but not limited to, Alzheimer's disease, Parkinson's disease or amyotrphic lateral sclerosis (ALS).

In some embodiments, the method comprises administration of a composition comprising a therapeutically effective amount of dihydro-lipoic acid or R-dihydro-lipoic acid to the subject diagnosed with at least one of ulcerative colitis, systemic lupus erythematosus (SLE), sepsis, post-sepsis syndrome, diabetes, Alzheimer's disease, Parkinson's disease, amyotrphic lateral sclerosis (ALS), autism, psoriasis, cataracts, and atrial fibrillation.

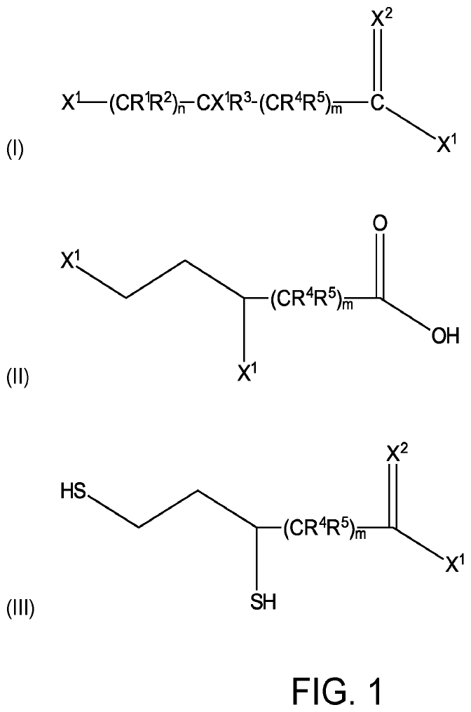

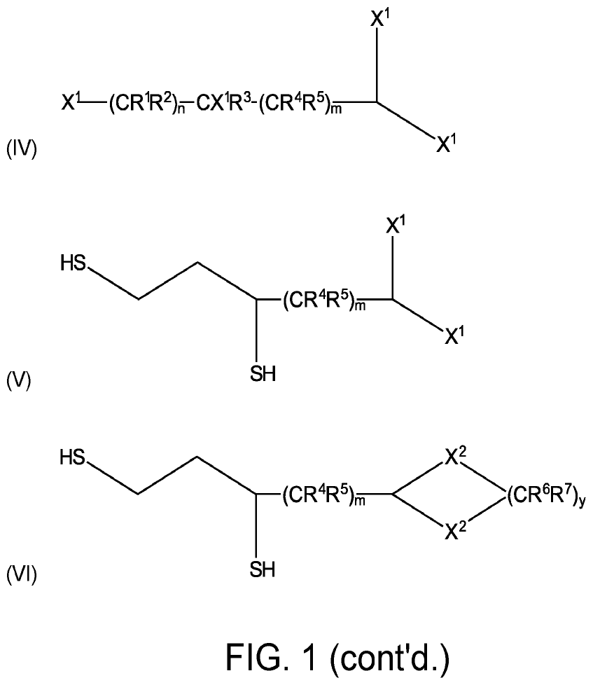

As noted herein, in some embodiments, the method includes administering a composition comprising a derivative of dihydro-lipoic acid. In this context, the term “derivative” refers to a compound having a structure that is derived from dihydro-lipoic acid or R-dihydro-lipoic acid, including compounds derived from dihydro-lipoic acid or R-dihydro-lipoic acid via a structural modification to the eight-carbon chain of dihydro-lipoic acid or to the one or more functional groups of dihydro-lipoic acid (i.e., the thiols and carboxylic acid). In some embodiments, the scope of the term “derivative of dihydro-lipoic acid” does not include dihydro-lipoic acid. In some embodiments, the composition comprises of dihydro-lipoic acid and derivatives of dihydro-lipoic acid according to Formulae I-VI, as shown in FIG. 1.

In some embodiments, the composition comprises derivatives of dihydro-lipoic acid according to Formula I, wherein: X¹ is selected from —SH, —OH, —SR⁸, —OR⁸; X² is selected from O or S; R¹, R², R³, R⁴, and R⁵ are independently selected from H, alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; R⁸ is selected from alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; n=0-3; and m=0-12.

In some embodiments, the composition comprises derivatives of dihydro-lipoic acid according to Formula II, wherein: X¹ is selected from —SH, —OH, —SR⁸, —OR⁸; R⁴ and R⁵ are independently selected from H, alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; R⁸ is selected from alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; m=0-12; and any unlabeled points in the structure represent a carbon atom substituted by one or more hydrogen atoms.

In some embodiments, the composition comprises derivatives of dihydro-lipoic acid according to Formula III, wherein: X¹ is selected from —SH, —OH, —SR⁸, —OR⁸; X² is selected from O or S; R⁴ and R⁵ are independently selected from H, alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; R⁸ is selected from alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; m=0-12; and any unlabeled points in the structure represent a carbon atom substituted by one or more hydrogen atoms.

In some embodiments, the composition comprises derivatives of dihydro-lipoic acid according to Formula IV, wherein: X¹ is selected from —SH, —OH, —SR⁸, —OR⁸; R¹, R², R³, R₄, and R⁵ are independently selected from H, alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; R⁸ is selected from alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; n=0-3; m=0-12; and any unlabeled points in the structure represent a carbon atom substituted by one or more hydrogen atoms.

In some embodiments, the composition comprises derivatives of dihydro-lipoic acid according to Formula V, wherein: X¹ is selected from —SH, —OH, —SR⁸, —OR⁸; R⁴ and R⁵ are independently selected from H, alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; R⁸ is selected from alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; m=0-12; and any unlabeled points in the structure represent a carbon atom substituted by one or more hydrogen atoms.

In some embodiments, the composition comprises derivatives of dihydro-lipoic acid according to Formula VI, wherein: X¹ is selected from —SH, —OH, —SR⁸, —OR₈; X² is selected from O or S; R⁴, R⁵, R⁶, and R⁷ are independently selected from H, alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; R⁸ is selected from alkyl, alkenyl, alkynyl, aryl, heteroaryl, aralkyl, alkylaryl, cycloalkyl, cylcoalkenyl, halogen, alkynylalkyl, heteroalkyl, hydroxyalkyl, acyl, aroyl, alkoxy, aryloxy, aralkyloxy, alkylthio, alkoxycarbonyl, aryloxycarbonyl, aralkoxycarbonyl, alkylsulfonyl, and arylsulfonyl; m=0-12; y=1-3; and any unlabeled points in the structure represent a carbon atom substituted by one or more hydrogen atoms.

In some embodiments, the scope of Formulae I-VI, independent of one another, does not include dihydro-lipoic acid (also known as 6,8-Bis(sulfanyl)octanoic acid).

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

As used herein, the terms “optional” or “optionally” mean that the subsequently described event or condition may but need not occur, and that the description includes instances where the event or condition occurs and instances in which it does not. The term “optionally substituted” means optional substitution with the specified groups, radicals or moieties; i.e., the named substituent may be present but need not be present.

“Alkyl” means an aliphatic hydrocarbon group which may be straight or branched and comprising about 1 to about 20 carbon atoms in the chain. Exemplary alkyl groups contain about 1 to about 12 carbon atoms in the chain, or about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. A substituted alkyl group may be substituted by one or more substituents, which may be the same or different, each independently selected from halo, alkyl, aryl, cycloalkyl, cyano, hydroxy, alkoxy, alkylthio, amino, —NH(alkyl), —NH(cycloalkyl), —N(alkyl)₂, carboxy, and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Exemplary alkenyl groups have about 2 to about 12 carbon atoms in the chain, or about 2 to about 6 carbon atoms in the chain. Branched means that one or more lower alkyl groups such as methyl, ethyl, or propyl, are attached to a linear alkenyl chain. “Lower alkenyl” means about 2 to about 6 carbon atoms in the chain which may be straight or branched. Non-limiting examples of suitable alkenyl groups include ethenyl, propenyl, n-butenyl, 3-methylbut-2-enyl, n-pentenyl, octenyl, and decenyl.

“Alkynyl” means an aliphatic hydrocarbon group containing at least one carbon-carbon triple bond and which may be straight or branched and comprising about 2 to about 15 carbon atoms in the chain. Exemplary alkynyl groups have about 2 to about 12 carbon atoms in the chain, or about 2 to about 4 carbon atoms in the chain. Branched means that one or more lower alkyl groups such as methyl, ethyl, or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more substituents which may be the same or different, and which are as defined herein. Non-limiting examples of suitable aryl groups include phenyl and naphthyl.

“Heteroaryl” means an aromatic monocyclic or multicyclic ring system comprising about 5 to about 14 ring atoms, or about 5 to about 10 ring atoms, in which one or more of the ring atoms is an element other than carbon, for example nitrogen, oxygen or sulfur, alone or in combination. Exemplary heteroaryls contain about 5 to about 6 ring atoms. The “heteroaryl” can be optionally substituted by one or more substituents, which may be the same or different. The prefix aza, oxa or thia before the heteroaryl root name means that at least a nitrogen, oxygen or sulfur atom, respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Aralkyl” or “arylalkyl” means an aryl-alkyl-group in which the aryl and alkyl are as previously described. Exemplary aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Exemplary alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, or about 5 to about 10 carbon atoms. Exemplary cycloalkyl rings contain about 5 to about 7 ring atoms. The cycloalkyl can be optionally substituted with one or more substituents which may be the same or different. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkenyl” means a non-aromatic mono or multicyclic ring system comprising about 3 to about 10 carbon atoms, or about 5 to about 10 carbon atoms, which contains at least one carbon-carbon double bond. Exemplary cycloalkenyl rings contain about 5 to about 7 ring atoms. The cycloalkenyl can be optionally substituted with one or more substituents, which may be the same or different. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Halogen” (or “halo”) means fluorine, chlorine, bromine, or iodine. Exemplary halogens include fluorine, chlorine, and bromine.

“Alkynylalkyl” means an alkynyl-alkyl-group in which the alkynyl and alkyl are as previously described. Exemplary alkynylalkyls contain a lower alkynyl and a lower alkyl group. Non-limiting examples of suitable alkynylalkyl groups include propargylmethyl. The bond to the parent moiety is through the alkyl.

“Heteroaralkyl” means a heteroaryl-alkyl-group in which the heteroaryl and alkyl are as previously described. Exemplary heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Exemplary hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. Exemplary acyls contain a lower alkyl. Non-limiting examples of suitable acyl groups include formyl, acetyl and propanoyl. The bond to the parent moiety is through the carbonyl carbon.

“Aroyl” means an aryl-C(O)— group in which the aryl group is as previously described. Non-limiting examples of suitable groups include benzoyl and 1-naphthoyl. The bond to the parent moiety is through the carbonyl carbon.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. A non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl carbon.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl carbon.

“Aralkoxycarbonyl” means an aralkyl-O—C(O)— group. A non-limiting example of a suitable aralkoxycarbonyl group is benzyloxycarbonyl. The bond to the parent moiety is through the carbonyl carbon.

“Alkylsulfonyl” means an alkyl-S(O₂)— group. Exemplary groups are those in which the alkyl group is lower alkyl. The bond to the parent moiety is through the sulfonyl sulfur.

“Arylsulfonyl” means an aryl-S(O₂)— group. The bond to the parent moiety is through the sulfonyl sulfur.

When any variable (e.g., aryl, heterocycle, R², etc.) occurs more than one time in any constituent or in the above identified compounds, its definition on each occurrence is independent of its definition at every other occurrence.

Prodrugs and solvates of the compounds of Formulae I-VI are also contemplated herein. The term “prodrug,” as employed herein, denotes a compound that is a drug precursor which, upon administration to a subject, undergoes chemical conversion by metabolic or chemical processes to yield dihydro-lipoic acid or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference in their entireties.

In some embodiments, if a compound of Formulae I-VI or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₅)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 8-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino (C₂-C₃)alkyl, and the like.

In some embodiments, if a compound of Formulae I-VI contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino (C₁-C₄)alkanyl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

In some embodiments, one or more compounds of Formulae I-VI may exist in unsolvated as well as solvated forms with pharmaceutically acceptable solvents such as water, ethanol, and the like, and it is intended that both solvated and unsolvated forms are included. “Solvate” means a physical association of a compound of Formulae I-VI with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of illustrative solvates include ethanolates, methanolates, and the like. “Hydrate” is a solvate wherein the solvent molecule is water (H₂O).

In some embodiments, one or more compounds of Formulae I-VI may optionally be converted to a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTech., 5(1), article 12 (2004); and A. L. Bingham et al., Chem. Commun., 603-604 (2001). A typical, non-limiting, process involves dissolving the compound in desired amounts of the desired solvent (organic or water or mixtures thereof) at a higher than ambient temperature, and then cooling the solution at a rate sufficient to form crystals which are then isolated by standard methods. Analytical techniques such as, for example infrared (IR) spectroscopy, show the presence of the solvent (or water) in the crystals as a solvate (or hydrate).

Metabolic conjugates, such as glucuronides and sulfates which can undergo reversible conversion to the compounds of Formulae I-VI are contemplated.

“Effective amount” or “therapeutically effective amount” is meant to describe an amount of compound or a composition of Formulae I-VI effective in producing the desired therapeutic, ameliorative, inhibitory, or preventative effect.

The terms “purified,” “in purified form” or “in isolated and purified form,” as used herein, for a compound refers to the physical state of said compound after being isolated from a synthetic process (e.g., from a reaction mixture), or natural source or combination thereof. The term “purified,” “in purified form” or “in isolated and purified form” for a compound refers to the physical state of said compound after being obtained from a purification process or processes described herein or known to one skilled in the art (e.g., chromatography, distillation, recrystallization), in sufficient purity to be characterizable by standard analytical techniques known to one skilled in the art. In some embodiments, a “purified form” refers to 90% pure, 95% pure, 97% pure, or 99% pure, as determined by standard analytical techniques known to one skilled in the art.

In some embodiments, the compounds of Formulae I-VI can form salts. Reference to a compound of Formulae I-VI herein is understood to include reference to salts thereof, unless otherwise indicated. The term “salt(s),” as employed herein, denotes acidic salts formed with inorganic and/or organic acids, as well as basic salts formed with inorganic and/or organic bases. In addition, when a compound of Formulae I-VI contains both a basic moiety, such as, pyridine or imidazole, and an acidic moiety, such as, a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of Formulae I-VI or may be formed, for example, by reacting a compound of Formulae I-VI with an amount of acid or base, such as an equivalent amount, in a medium such as one in which the salt precipitates or in an aqueous medium followed by lyophilization.

Examples of pharmaceutically acceptable salts include, but are not limited to, mineral or organic acid salts of basic residues such as amines, and alkali or organic salts of acidic residues such as carboxylic acids, and the like. The pharmaceutically acceptable salts include conventional non-toxic salts or quaternary ammonium salts of the parent compound formed, for example, from inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., (1985), 1418, the disclosure of which is incorporated herein by reference.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by S. Berge et al., J. of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and The Orange Book (Food and Drug Administration, Washington, D.C.). These disclosures are incorporated herein by reference.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine, and the like. Basic nitrogen-containing groups may be quarternized with agents such as lower alkyl halides (e.g., methyl, ethyl, and butyl chlorides, bromides, and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts, and all acid and base salts are considered equivalent to the free forms of the corresponding compounds for purposes of this disclosure.

As to any of the above groups, which contain one or more substituents, it is understood, of course, that such groups do not contain any substitution or substitution patterns that are sterically impractical and/or synthetically non-feasible.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons or sulfurs on various substituents, including enantiomeric forms (which may exist even in the absence of asymmetric carbons), rotameric forms, atropisomers, and diastereomeric forms, are contemplated. For example, if a compound of Formulae I-VI incorporates a double bond or a fused ring, both the cis- and trans-forms, as well as mixtures, are embraced within the scope of the disclosure. Individual stereoisomers of the compounds may, for example, be substantially free of other isomers, or may be admixed, for example, as racemates or with all other, or other selected, stereoisomers. The chiral centers of the compounds can have the S or R configuration as defined by the IUPAC 1974 Recommendations. The use of the terms “salt,” “solvate,” “prodrug,” and the like, is intended to equally apply to the salt, solvate, and prodrug of enantiomers, stereoisomers, rotamers, tautomers, racemates, or prodrugs of the compounds.

Diastereomeric mixtures can be separated into their individual diastereomers based on their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formulae I-VI may be atropisomers (e.g., substituted biaryls). Enantiomers can also be separated by use of chiral HPLC column.

Polymorphic forms of the compounds of Formulae I-VI, and of the salts, solvates and prodrugs of the compounds of Formulae I-VI, are intended to be included.

In some embodiments, isotopically labelled compounds, which are identical to those recited herein, but for the fact that one or more atoms are replaced by an atom having an atomic mass or mass number different from the atomic mass or mass number usually found in nature. Examples of isotopes that can be incorporated into compounds include isotopes of hydrogen, carbon, nitrogen, oxygen, phosphorus, fluorine, and chlorine, such as 2H, 3H, 13C, 14C, 15N, 18O, 17O, 31P, 32P, 35S, 18F, and 36Cl, respectively.

Certain isotopically labelled compounds of Formulae I-VI (e.g., those labeled with 3H and 14C) are useful in compound and/or substrate tissue distribution assays. Tritiated (i.e., 3H) and carbon-14 (i.e., 14C) isotopes are particularly preferred for their ease of preparation and detectability. Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Isotopically labelled compounds of Formulae I-VI can generally be prepared by following procedures analogous to those disclosed in the Schemes and/or in the Examples hereinbelow, by substituting an appropriate isotopically labelled reagent for a non-isotopically labelled reagent.

In some embodiments, the method comprises administering a composition comprising a derivative of dihydro-lipoic acid to a subject in need thereof. In some embodiments, the composition comprises a compound according to Formulae I-VI, pharmaceutically acceptable salts thereof, or a combination thereof. In some embodiments, the composition comprises a compound according to Formulae I-VI or a pharmaceutically acceptable salt thereof. In some embodiments, the method does not include the co-administration of an enema composition to the subject. In some embodiments, the method comprises administration of a composition comprising a therapeutically effective amount of a compound according to Formulae I-VI to the subject during the induction phase of ulcerative colitis.

The compounds of Formulae I-VI have pharmacological properties; in particular, the compounds of Formulae I-VI are capable of entering a cell and reacting with hydrogen peroxide.

Exemplary compounds of Formulae I-VI are provided in FIG. 2. In some embodiments, the method comprises administering a composition comprising dihydro-lipoic acid (compound a) or a derivative of dihydro-lipoic acid (compounds (b)-(h)) to a subject in need thereof.

The natural function of ALA is to bind and transfer acyl groups to successive enzymatic active sites among the subunits of each enzyme complex. In this process of acyl transfer, ALA is reduced to dihydro-lipoic acid and subsequently re-oxidized back to ALA by its attached cognate enzyme which readies it for the next acyl transfer. ALA has a high degree of bioavailability after oral administration and exhibits both lipid and water solubility. This allows its distribution to both intra and extra cellular compartments.

### Compositions.

In some embodiments, the present inventive concept concerns not only administration of an active component, but also the active component. In some embodiments, compositions provided by the present inventive concept include dihydro-lipoic acid, which includes a derivative, pharmaceutically acceptable salt, metabolite, solvate, prodrug, enantiomer, or polymorph of the dihydro-lipoic acid; a medium-chain triglyceride and/or long-chain triglyceride; a protein derived from collagen; a sugar alcohol; water; at least one glycerophospholipid; at least one fat soluble vitamin; a silicon-based compound; optionally a preservative; and optionally a flavoring. In some embodiments, the medium-chain triglyceride and/or long-chain triglyceride is MCT oil, olive oil or coconut oil. In some embodiments, the protein derived from collagen is gelatin. In other embodiments, the sugar alcohol is glycerin. In further embodiments, a combination of glycerophospholipids is lecithin as described herein, including soy lecithin or sunflower lecithin. In some embodiments, the at least one fat soluble vitamin is Vitamin E and/or ascorbyl palmitate. According to some embodiments, the silicon-based compound is silicon dioxide. In some embodiments, the preservative is natural preservative such as rosemary extract. In still other embodiments, the flavoring is a natural flavoring such as carob. In some embodiments, the composition includes a medicinal herb. Such medicinal herbs include, but are not limited to, ginger, peppermint, turmeric, aloe vera, cinnamon, fennel, garlic, cannabis, Delta-9-tetrahydrocannabinol (THC), triphala, slippery elm, licorice, chamomile, clove, bearberry, rose, flower, marjoram, Lady's mantle, allspice, blackcurrant, oregano, mint, thyme, milk thistle, and silymarin.

In further embodiments of the present inventive concept, the composition comprises N-acetyl-L-cysteine (N-A-C), and/or L-glutamine. In some embodiments, the composition comprises compounds that inhibit tissue necrosis factor (TNF). TNF inhibitory compounds contemplated include resveratrol, stinging nettle leaf extract, and berberine. In some embodiments, the composition includes compounds that directly neutralize hydrogen peroxide (e.g., calcium pyruvate), compounds that help protect the proteins from oxidation and degradation (e.g., L-carnosine), and/or compounds that are protective of nucleic acids (e.g., calcium D-glucarate). The composition can also optionally comprise one or more of the following: selenium, vitamin B-2 (riboflavin), vitamin B-12, folic acid, and biotin.

In some embodiments, the composition comprises one or more carriers, excipients, diluents, stabilizers, lubricants, glidants, moisturizers, penetration enhancers, and/or solubilizers, such as those used in compositions for oral administration. In some embodiments, diluents and excipients such as fillers, extenders, binders, wetting agents, disintegrants, surfactants, or coloring agents can be used.

In some embodiments, the composition comprises one or more herb components, plant components, and/or extracts. In such embodiments, the herb component, plant components, and/or extracts may include one or more flavonoids, terpenes, terpenoids, amino acids, proteins, sugars, enzymes, fatty acids, ester, and/or other organic compounds. In some embodiments, the composition includes an herb such as ginger, peppermint, turmeric, aloe vera, cinnamon, fennel, garlic, cannabis, Delta-9-tetrahydrocannabinol (THC), triphala, slippery elm, licorice, chamomile, clove, bearberry, rose, flower, marjoram, Lady's mantle, allspice, blackcurrant, oregano, mint, thyme, milk thistle, and silymarin.

Specific examples of carriers, excipients, and diluents that can be included in the composition may include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, talc, magnesium stearate, methylhydroxybenzoate, propylhydroxybenzoate, mineral oil, and the like. Additional examples of carriers include oils, including, e.g., medium-chain triglyceride (MCT), coconut oil, olive oil, etc.

“Diluent” refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

“Disintegrants” refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth, and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

“Binders” refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin, and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

“Lubricant” is meant to describe a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water-soluble lubricants such as sodium chloride, sodium benzoate, sodium acetate, sodium oleate, polyethylene glycols and D- or L-leucine. Lubricants are usually added at the very last step before compression since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

“Glidants” means materials that prevent caking and improve the flow characteristics of granulations, so that flow is smooth and uniform. Suitable glidants include silicon dioxide and talc. The amount of glidant in the composition can range from about 0.1% to about 5% by weight of the total composition, preferably from about 0.5 to about 2% by weight.

“Coloring agents” refers to excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Specific examples of preservatives that may be included in the compositions include rosemary extract, neem oil, honey, lemon, ascorbyl palmitate, vitamin E, vitamin C, butylatedhydroxyanisole (BHA), butylatedhydroxytoluene (BHT), benzoates, sorbates, propionates, nitrites, sulfites, citric acid, polyphosphates, disodium ethylenediamine tetraacetic acid (EDTA), and the like. Moreover, in some embodiments, the preservative may also serve as an antioxidant and/or an antimicrobial.

In some embodiments, the composition comprises a suitable lipid or phospholipid component. A “phospholipid” describes compounds comprising a lipid moiety and a phosphate moiety. A suitable phospholipid includes phospholipids belonging to the glycerophospholipid class (also known as phosphoglycerols or as diacylglyceride phosphates), including, but not limited to, phosphatidic acid (phosphatidate) (PA), phosphatidylethanolamine (cephalin) (PE), phosphatidylcholine (lecithin) (PC), phosphatidylserine (PS), and phosphoinositides including, for example, phosphatidylinositol (PI), phosphatidylinositol phosphate (PIP), phosphatidylinositol bisphosphate (PIP2) and phosphatidylinositol triphosphate (PIP3); or those belonging to the phosphosphingolipids class (which are derived from sphongosine), including, but not limited to, ceramide phosphorylcholine (sphingomyelin) (SPH), ceramide phosphorylethanolamine (Cer-PE), and ceramide phosphorylglycerol.

In some embodiments, the composition comprises a lecithin component. In such embodiments, the lecithin may be synthetic or obtained from a natural product. In some embodiments, the lecithin is obtained from egg yolk, marine foods, soybeans, milk, rapeseed, cottonseed, or sunflower oil. In some embodiments, the composition comprises a lecithin alternative. In such embodiments, the lecithin alternative is stearic, linoleic, or palmitic acid.

### Formulations.

In some embodiments, the composition is formulated in the form of oral preparations such as powders, granules, tablets, capsules, suspensions, emulsions, oral gels, syrups, liquids, aerosols, or the like, according to an existing method. In some embodiments, the composition is formulated for transdermal administration.

A solid formulation for oral administration may include tablets, pills, powders, granules, capsules, gummies, and the like. In such embodiments, the solid formulation may include at least one excipient such as starch, calcium carbonate, sucrose or lactose, and gelatin, in addition to the composition. In addition to simple excipients, lubricants such as magnesium stearate and talc may also be included.

“Tablet” is meant to describe a compressed or molded solid dosage form containing the active ingredients with suitable diluents. The tablet can be prepared by compression of mixtures or granulations obtained by wet granulation, dry granulation or by compaction.

“Oral gels” is meant to describe the active ingredients dispersed or solubilized in a hydrophilic semi-solid matrix.

“Gummy,” “Gummies,” or “Chewable gel” compositions refer to compositions having the active ingredient incorporated into a base ingredient composed of gelatin, pectin, vegan gel, agar, and the like, and often including flavoring and/or coloring agents.

“Powders for constitution” refers to powder blends containing the active ingredients and suitable diluents which can be suspended in water or juices.

“Capsule” is meant to describe a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers, and preservatives.

In particular embodiments, the composition may be formulated for oral administration wherein the oral preparation is in the form of a capsule.

A liquid formulation for oral administration may include suspensions, solutions, emulsions, syrups, and the like. In such embodiments, various excipients such as wetting agents, sweetening agents, fragrances, preservatives, and the like may be included in addition to water and liquid paraffin which are commonly employed simple diluents. In particular embodiments, the composition may be formulated for oral administration wherein the oral preparation is in the form of a liquid.

In some embodiments, the composition is formulated in the form of a modified-release capsule. As used herein, the term “modified-release” formulation or dosage form includes pharmaceutical preparations that achieve a desired release of the drug from the formulation. A modified-release formulation can be designed to modify the manner in which the active ingredient is exposed to the desired target. For example, a modified-release formulation can be designed to focus the delivery of the active agent entirely in the distal large intestine, beginning at the cecum, and continuing through the ascending, transverse, and descending colon, and ending in the sigmoid colon. Alternatively, for example, a modified-release composition can be designed to release to focus the delivery of the drug in the proximal small intestine, beginning at the duodenum and ending at the ileum. In still other examples, the modified-release formulations can be designed to begin releasing active agent in the jejunum and end their release in the transverse colon. The possibilities and combinations are numerous, and are clearly not limited to these examples. The term “modified-release” encompasses “extended-release” and “delayed-release” formulations, as well as formulations having both extended-release and delayed-release characteristics. An “extended-release” formulation can extend the period over which a drug is released or targeted to the desired site. A “delayed-release” formulation can be designed to delay the release of the pharmaceutically active compound for a specified period. Such formulations are referred to herein as “delayed-release” or “delayed-onset” formulations or dosage forms. Modified-release formulations of the present disclosure include those that exhibit both a delayed- and extended-release, e.g., formulations that only begin releasing after a fixed period of time or after a physicochemical change has occurred, for example, then continue releasing over an extended period. By contrast, the term “immediate-release formulation,” as used herein, is meant to describe those formulations in which more than about 50% of active ingredient is released from the dosage form in less than about 2 hours.

In some embodiments, the composition is provided in an orally administered form, such as a capsule, that dissolves in a subject's stomach and/or small intestine. In another embodiment, the composition is provided in an orally administered capsule that delays dissolving until it is present in the colon. Delayed dissolution dosage forms include pH-dependent capsules and coatings that only dissolve at the pH associated with the colonic environment. Examples of pH-dependent materials include, but are not limited to, methyl methacrylate, methacrylic acid and/or ethyl acrylate polymers, including for example, ammonio methacrylate copolymer. Other dosage forms for delivery of a composition to the colon include, for example, time-dependent delivery systems, pressure-dependent delivery systems, and bacterial-dependent systems. Also contemplated are dosage forms that utilize oxidation potential-dependent systems.

“Transdermal” refers generally to passage of an agent across the skin layers. For example, the term “transdermal” may refer to delivery of an agent (e.g., a drug) through the skin to the local tissue or systemic circulatory system without substantial cutting or penetration of the skin, such as cutting with a surgical knife or piercing the skin with a hypodermic needle. Transdermal agent delivery includes delivery via passive diffusion. In some embodiments, the transdermal composition may be part of, for example, a transdermal patch, ointment, cream, gel, lotion or other transdermal solution or suspension. For example, for transdermal delivery, a transdermal patch that includes a disclosed composition is contemplated, and may include a single layer adhesive patch, a multi-layer and adhesive patch, a reservoir patch, a matrix patch, a microneedle patch or an iontophoretic patch, which typically requires applying a direct current. In some embodiments, transdermal patches may be adapted for sustained or modified release.

In some embodiments, the composition may be administered orally as a gummy or chewable gel including the dihydro-lipoic acid including derivatives, pharmaceutically acceptable salts, metabolites, solvates, prodrugs, enantiomers, or polymorphs of the dihydro-lipoic acid. The formulation may include color and/or flavoring agents.

In some embodiments, intravenous or (IV) formulations may be suitable. For example, patients that are hospitalized with severe ulcerative colitis may be administered dihydro-lipoic acid, which includes derivatives, pharmaceutically acceptable salts, metabolites, solvates, prodrugs, enantiomers, or polymorphs of the dihydro-lipoic acid. Specifically, the IV administration may be warranted for a subject suffering from acute severe ulcerative colitis requiring emergency medical attention.

Additionally, patients who are hospitalized for systemic lupus erythematosus (SLE), sepsis, post-sepsis syndrome may receive IV administration of dihydro-lipoic acid, which includes derivatives, pharmaceutically acceptable salts, metabolites, solvates, prodrugs, enantiomers, or polymorphs of the dihydro-lipoic acid.

In some embodiments, the composition is formulated as a suppository. As the active compound typically has a pH of about 4.5, a therapeutically effective amount of buffered dihydro-lipoic acid including derivatives, pharmaceutically acceptable salts, metabolites, solvates, prodrugs, enantiomers, or polymorphs of the dihydro-lipoic acid, can be administered directly to the rectum as a suppository to treat or prevent the conditions described herein, and particularly, ulcerative colitis. Such buffering agents and/or salt forms of the active compound are described herein.

In some embodiments, the formulation, such as capsule, liquid, gummy, IV, and intrarectal, specifically include buffered dihydro-lipoic acid including its derivatives, and/or pharmaceutically acceptable salts of dihydro-lipoic acid including its derivatives. Such buffering agents and/or salt forms of the active compound are described herein.

### Dosage.

In some embodiments, the oral component is administered at least once daily. In some embodiments, the oral component is administered twice daily. In some other embodiments, the oral component is administered every other day. In still some embodiments, a composition comprises an agent which directly, or indirectly through an intermediary mechanism, acts as an intracellular or extracellular reducing agent and neutralizes or otherwise prevents the formation of intra or extracellular hydrogen peroxide or oxygen radicals.

The dose of the active compound(s) in the composition is not particularly limited. The actual dosage employed may be varied depending upon the requirements of the patient and the severity of the condition being treated. Determination of the proper dosage regimen for a particular situation is within the skill of the art. For convenience to the subject, the total daily dosage may be divided and administered in portions during the day as required. The amount and frequency of administration of the compounds and/or the pharmaceutically acceptable salts thereof will be regulated according to the judgment of the attending clinician considering such factors as age, condition, and size of the patient as well as the severity of the symptoms being treated.

In some embodiments, the composition is prepared in a unit dosage form. In such form, the preparation is subdivided into suitably sized unit doses containing appropriate quantities of the active component, e.g., an effective amount to achieve the desired purpose. The quantity of active compound in a unit dose of preparation may be varied or adjusted from about 1 mg to about 1000 mg. In some embodiments, the composition includes the compound in an amount ranging from about 25 mg to about 1000 mg, from about 50 mg to about 750 mg, from about 100 mg to about 600 mg, or from about 200 mg to about 500 mg. In some embodiments, the composition comprises about 25 mg, or about 50 mg, or about 100 mg, or about 200 mg of the compound. These amounts may be administered one or more times daily (e.g., 40 mg or 150 mg taken twice daily). In some embodiments, the compound is administered every day or every other day. In some embodiments, the compound is taken every day or every other day for a period of about 30 days to 180 days or more. In some embodiments the compound is taken 30 days, 60 days, 90 days, 120 days, 150 days or 180 days.

In some embodiments, the dose of the compound may be appropriately selected depending on the condition and body weight of the subject, the severity of disease or condition, the drug form, and the period of administration. A typical recommended daily dosage regimen for oral administration can range from about 1 mg/day to about 1000 mg/day, or from about 1 mg/day to 200 mg/day, in two to four divided doses. Alternatively, the dosage regimen is every other day. In some embodiments, the compound is administered at 5 to 10 mg/kg.

### EXAMPLES

The following examples are provided to aid in the understanding of the present disclosure, the true scope of which is set forth in the appended claims. One of skill in the art would appreciate that modifications can be made in the procedures set forth without departing from the spirit of the disclosure.

### Example 1

A composition comprising 150 mg of R-dihydro-lipoic acid, MCT oil, gelatin, glycerin, water, soy lecithin, vitamin E, silicon dioxide, rosemary extract, ascorbyl palmitate, and carob.

### Example 2

A composition comprising 50 mg of R-dihydro-lipoic acid, olive oil, gelatin, glycerin, water, soy lecithin, vitamin E, silicon dioxide, rosemary extract, ascorbyl palmitate, and carob.

### Example 3

A composition comprising 40 mg of R-dihydro-lipoic acid, coconut oil, gelatin, glycerin, water, sunflower lecithin, vitamin E, silicon dioxide, rosemary extract, ascorbyl palmitate, and carob.

### Example 4

A subject (41-year-old female) diagnosed with refractory unresponsive ulcerative colitis, who had for about 25 years dealt with bouts of rectal bleeding, was treated with the composition comprising 150 mg of R-dihydro-lipoic acid, MCT oil, gelatin, glycerin, water, soy lecithin, vitamin E, silicon dioxide, rosemary extract, ascorbyl palmitate, and carob according to a once every other day schedule for 2 months. The subject reported her bouts of rectal bleeding had stopped after about 7 days. Since treatment began, the subject has reported that no bouts of rectal bleeding have occurred, that she is completely asymptomatic, and has normal stool calprotectin levels. Calprotectin is a protein inside neutrophils that is released when white blood cells enter the colon to cause colitis (colonic inflammation). Normal stool calprotectin levels are indicative of no colonic inflammation. During the period of treatment, the subject did not receive any enema treatments or any other treatments for ulcerative colitis.

It has been surprisingly found that the method of administering an oral composition comprising a compound of Formulae I-VI without the co-administration of an enema composition or an immunosuppressive agent provides an effective treatment for ulcerative colitis. It has further been surprisingly found that the treatment of ulcerative colitis does not need to be divided into a first phase of inducing the ulcerative colitis into remission (e.g., using highly potent immunosuppressive agent for about 6 weeks to reduce colonic (colitis) inflammation) and a second phase of maintaining remission (e.g., using less potent immunosuppressive agent indefinitely to maintain colonic inflammation at a low level).

According to the methods of the present disclosure, administration of a composition including an active compound (e.g., R-dihydro-lipoic acid), without the administration of further therapeutic agents and without switching to a different therapeutic agent, will eliminate toxic amounts of intracellular and extracellular colonocyte hydrogen peroxide thereby reducing inflammation associated with ulcerative colitis. The methods do not include administering potent immunosuppressive agents, which can cause significant and serious adverse effects.

### Example 5

In various embodiments, a method of administering an oral composition comprising a compound of Formulae I-VI to a subject in need thereof provides an effective treatment for systemic lupus erythematosus (SLE), sepsis, and/or post-sepsis syndrome. According to the methods of the present disclosure, the administration of a composition comprising an active compound (e.g., R-dihydro-lipoic acid) will eliminate toxic amounts of intracellular and extracellular hydrogen peroxide, which has been determined to be a cause of symptoms (e.g., inflammation) associated with systemic lupus erythematosus (SLE), sepsis, and/or post-sepsis syndrome.

In some embodiments, the method comprises administrating a composition formulated for oral administration to a subject in need thereof. In some embodiments, the oral composition is administered every third day. In some embodiments, the oral composition is administered every other day. In some embodiments, the oral composition is administered at least once daily. In some embodiments, the oral composition is administered twice daily.

In some embodiments, the method does not include the coadministration of an immunosuppressive agent, which can cause significant and serious adverse effects. In such embodiments, the administration of a composition comprising an active compound of Formulae I-VI to a subject in need thereof will eliminate toxic amounts of intracellular and extracellular hydrogen peroxide without the administration of further therapeutic agents and without switching to a different therapeutic agent during the treatment schedule.

In some embodiments, the administration of a composition comprising an active compound of Formulae I-VI to a subject in need thereof can cause a direct reaction of hydrogen peroxide present in the subject's blood and act to lower the hydrogen peroxide blood levels. The reduction in hydrogen peroxide blood levels can decrease intracellular hydrogen peroxide and prevent, or reduce the risk of, mitochondrial DNA damage that can persist indefinitely after a subject is discharged from an ICU, causing post-sepsis syndrome. Thus, a composition comprising an active compound may be administered while a subject is suffering from sepsis in an ICU and continued indefinitely after their discharge from the ICU.

Administration of a composition comprising an active compound of Formulae I-VI (e.g., dihydro-lipoic acid, R-dihydro-lipoic acid) to a subject in need thereof may increase lymphocyte reductive capacity and prevent, or reduce the risk of, apoptosis and the activation of the subject's immune system. As a result, acute attacks of lupus may be mitigated. For subjects experiencing an acute attack of lupus, one immediate goal for treatment is to stop lymphocytes from producing cytokines. To that end, the co-administration of cyclophosphamide and a composition comprising an active compound of Formulae I-VI may decrease the severity and length of the acute attack by preventing, or reducing the risk of, further apoptosis. Thus, a composition comprising an active compound of Formulae I-VI (e.g., dihydro-lipoic acid) may be administered for the treatment of acute attacks and for treating lupus generally.

Exemplary embodiments of the methods are described above in detail. The methods are not limited to the specific embodiments described herein, but rather, components of the compositions and/or steps of the method may be utilized independently and separately from other components and/or steps described herein. This written description uses examples to disclose the present embodiments, including the best mode, and to enable any person skilled in the art to practice the present embodiments, including making and using any compositions or performing any methods. The patentable scope of the present embodiments is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

1. A method of treating or preventing (a) ulcerative colitis, systemic lupus erythematosus (SLE), sepsis, or post-sepsis syndrome; (b) a neurodegenerative disorder; (c) the risk or incidence of colectomy or ulcerative colitis-associated colorectal cancer and/or (d) a condition associated with an excessive buildup of hydrogen peroxide
comprising administering a therapeutically effective amount of dihydro-lipoic acid, or a derivative, pharmaceutically acceptable salt, metabolite, solvate, prodrug, enantiomer, or polymorph of the dihydro-lipoic acid, to a subject in need of such treatment.

2. The method of claim 1, wherein the dihydro-lipoic acid, or a derivative, pharmaceutically acceptable salt, metabolite, solvate, prodrug, enantiomer, or polymorph of the dihydro-lipoic acid is formulated for oral administration.

3. The method of claim 1, wherein the method does not include co-administration of the dihydro-lipoic acid, or a derivative, pharmaceutically acceptable salt, metabolite, solvate, prodrug, enantiomer, or polymorph of the dihydro-lipoic acid with an enema composition and/or an immunosuppressive agent.

4. The method of claim 1, wherein the method includes co-administration of the dihydro-lipoic acid, or a derivative, pharmaceutically acceptable salt, metabolite, solvate, prodrug, enantiomer, or polymorph of the dihydro-lipoic acid with sodium butyrate.

5. The method of claim 1, wherein the dihydro-lipoic acid, or a derivative, pharmaceutically acceptable salt, metabolite, solvate, prodrug, enantiomer, or polymorph of the dihydro-lipoic acid is administered to the subject during an induction phase of ulcerative colitis.

6. The method of claim 1, wherein the subject in need thereof suffers from moderate to severe ulcerative colitis or refractory unresponsive ulcerative colitis.

7. The method of claim 1, wherein the subject is asymptomatic for ulcerative colitis, and optionally, has histologic inflammatory features of ulcerative colitis.

8. (canceled)

9. The method of claim 1, wherein the dihydro-lipoic acid, or a derivative, pharmaceutically acceptable salt, metabolite, solvate, prodrug, enantiomer, or polymorph of the dihydro-lipoic acid is administered to the subject in an amount in a range from about 30 mg to 600 mg.

10. The method of claim 1, wherein the dihydro-lipoic acid, or a derivative, pharmaceutically acceptable salt, metabolite, solvate, prodrug, enantiomer, or polymorph of the dihydro-lipoic acid is administered to the subject once a day; or once every other day.

11. (canceled)

12. (canceled)

13. (canceled)

14. The method of claim 1, for reducing the risk or incidence of colectomy or ulcerative colitis-associated colorectal cancer, wherein the dihydro-lipoic acid, or a derivative, pharmaceutically acceptable salt, metabolite, solvate, prodrug, enantiomer, or polymorph of the dihydro-lipoic acid is administered to the subject in an amount in a range from about 30 mg to 600 mg.

15. (canceled)

16. The method of claim 1, wherein the neurodegenerative disorder is Alzheimer's disease, Parkinson's disease or amyotrphic lateral sclerosis (ALS).

17. (canceled)

18. The method of claim 1, wherein the condition associated with excessive buildup of hydrogen peroxide includes ulcerative colitis, systemic lupus erythematosus (SLE), sepsis, postsepsis syndrome, diabetes, Alzheimer's disease, Parkinson's disease, amyotrphic lateral sclerosis (ALS), autism, psoriasis, cataracts, and atrial fibrillation.

19. A composition comprising:
(a) dihydro-lipoic acid, or a derivative, pharmaceutically acceptable salt, metabolite, solvate, prodrug, enantiomer, or polymorph of the dihydro-lipoic acid;
(b) a medium-chain triglyceride and/or long-chain triglyceride;
(c) a protein derived from collagen;
(d) a sugar alcohol;
(e) water;
(f) at least one glycerophospholipid;
(g) at least one fat soluble vitamin;
(h) a silicon-based compound;
(i) optionally a preservative; and
(j) optionally a flavoring.

20. The composition of claim 19, wherein the medium-chain triglyceride and/or long-chain triglyceride is MCT oil, olive oil or coconut oil; the protein derived from collagen is gelatin; the sugar alcohol is glycerin; the at least one glycerophospholipid is soy lecithin or sunflower lecithin; the at least one fat soluble vitamin is Vitamin E and/or ascorbyl palmitate; the silicon-based compound is silicon dioxide; if present, the preservative is rosemary extract; and if present, the flavoring is carob.

21. The composition of claim 19, wherein the composition comprises R-dihydro-lipoic acid, MCT oil, gelatin, glycerin, lecithin, vitamin E, silicon dioxide, rosemary extract, ascorbyl palmitate, and carob.

22. The composition of claim 19, wherein the composition comprises R-dihydro-lipoic acid, olive oil, gelatin, glycerin, water, lecithin, vitamin E, silicon dioxide, rosemary extract, ascorbyl palmitate, and carob.

23. The composition of claim 19, wherein the composition comprises R-dihydro-lipoic acid, coconut oil, gelatin, glycerin, water, lecithin, vitamin E, silicon dioxide, rosemary extract, ascorbyl palmitate, and carob.

24. The composition of claim 19 further comprising sodium butyrate.

25. The composition of claim 19 for use as: (a) a medicament; (b) in the treatment of a condition associated with an excessive buildup of hydrogen peroxide or (c) in the treatment of ulcerative colitis, systemic lupus erythematosus (SLE), sepsis, post-sepsis syndrome, diabetes, Alzheimer's disease, Parkinson's disease, amyotrphic lateral sclerosis (ALS), autism, psoriasis, cataracts, and atrial fibrillation.

26. (canceled)

27. (canceled)

28. (canceled)

29. (canceled)

30. (canceled)
